(19) United States
(12) Patent Application Publication
(10) Pub. No.: US 2008/0188887 A1
(43) Pub. Date: Aug. 7, 2008

(54) Removable vascular filter and method of filter placement

(76) Inventor: Stanley Batiste, Las Vegas, NV (US)

Correspondence Address: WEIDE & MILLER, LTD., 7251 W. LAKE MEAD BLVD., SUITE 530, LAS VEGAS, NV, 89128, US

(21) Appl. No.: 12/069,369

(22) Filed: Feb. 7, 2008

Related U.S. Application Data
(60) Provisional application No. 60/900,378, filed on Feb. 7, 2007.
(60) Provisional application No. 60/904,547, filed on Mar. 2, 2007.

(51) Int. Cl. A61M 29/00

(52) U.S. Cl. 606/200

(57) ABSTRACT

A vascular filter system and method for implanting the same are disclosed. The filter system generally includes a filter housing and a filter element where the element is suspended within the housing by a plurality of filter holding members. The housing is held in place in a vein by a plurality of securing barbs which generally extend outward from the housing. The housing and its holding members may be bioabsorbable. In these embodiments, the filter element may include at least one filter barb to secure the element after the housing has been bioabsorbed. The filter system may be implanted by accessing a vein and inserting a deployment sheath containing the filter system. The deployment sheath is advanced to the proper location and a deployment member is used to release the filter system as the sheath is retracted. The deployment member and sheath may be removed.

### CROSS-REFERENCE TO RELATED APPLICATIONS

This application claims priority to U.S. Provisional Patent Application No. 60/900,378 filed on Feb. 7, 2007 titled REMOVABLE VASCULAR FILTER AND METHOD OF FILTER PLACEMENT and U.S. Provisional Patent Application No. 60/904,547 filed on Mar. 2, 2007 titled REMOVABLE VASCULAR FILTER AND METHOD OF FILTER PLACEMENT.

### BACKGROUND OF THE INVENTION

1. Field of the Invention

The present invention relates to vascular filters and, in particular to surgically implanted vascular filters which capture blood clots and prevent the clots from migrating to other regions of the circulatory system.

2. Related Art

Deep vein thrombosis (DVT) is a common problem and causes significant morbidity and mortality in the United States and throughout the world. DVT is caused when a blood clot forms in the deep veins of the legs. These blood clots typically occur due to slow or reduced blood flow through the deep veins such as when the patient cannot ambulate or otherwise efficiently circulate their blood. Another cause of inefficient circulation may be due to structural damage to the veins such as general trauma or subsequent to surgical procedures. Additionally, a blood clot may form in a deep vein due to a particular medical condition or a propensity for the patient to have a hypercoagubility state. For example, a woman on birth control who smokes has an increased risk of forming blood clots and is thus predisposed to DVT.

The result and clinical significance of DVT is when the clot breaks free from its location in the deep vein of the leg, the clot travels through the circulatory system and may eventually lodge in a location that is adverse to the patient's health. For example, the clot may dislodge from a location in the deep vein of the patient's leg and migrate through the heart and come to rest in the patient's lung causing a pulmonary embolism (PE) resulting in restricted circulation and possibly sudden death for the patient.

DVT & PE are currently prevented in several ways including anticoagulation therapy, thrombectomy, thrombolysis and inferior vena cava filter (IVC filter) placement. Anticoagulation therapy utilizes various medications that reduce the patient's propensity for forming blood clots. However, this form of therapy has the disadvantage that due to the patient's inability to form blood clots (due to the medication), there is an increased risk of excessive bleeding should the patient become injured, sustain surgical complications or develop internal hemorrhaging.

Thrombectomy is a procedure generally performed for treatment of a PE, in which a blood clot is extracted from the vein using a surgical procedure or by way of an intravenous catheter and a mechanical suction device. This form of treatment is risky and technically very difficult because the catheter has to be advanced through the vascular system and navigated to a specific location in order to extract the clot. Additionally, during a thrombectomy there is an increased risk of causing vascular damage due to the surgical procedure and use of various mechanical devices.

Thrombolysis is a medical technique that is generally performed for treatment of a PE, in which various medicines are infused into the region of the clot that subsequently causes the clot to dissolve. This form of treatment has the disadvantage that the medication may cause bleeding at other sites such as within the brain. For example, if a patient has previously had a tiny non-clinical stroke, the medication used in a thrombolysis may cause a previously healed vessel to bleed within the patients head.

IVC filters have been very successful in saving countless lives and are the mainstay of treatment in a population of patients predisposed to DVT and PE. IVC filter placement is usually conducted by surgically installing a filter in a large bore vein such as the inferior vena cava located in the patient's upper abdomen (See FIG. 1). The IVC filter is placed using a large bore catheter (introducer catheter) for delivery of the filter. There are several existing filters available for patient placement, some are permanent and some are removable for a limited time, after which the removable filter becomes permanent. In the case where a removable filter is utilized, additional complications arise when the filter must be removed. The known removable IVC filter is generally placed for a time period of a several weeks to a few months to prevent internal vascular scaring. However, removal of the current IVC filters is technically challenging and requires large bore access either through the internal jugular vein of the patient's neck or the common femoral vein.

The currently available IVC filters are all limited in their ability to be efficiently and safely removed from the patient after a predetermined time interval. In addition, although the current designs are approved for several weeks or months they can be extremely difficult to remove and do cause injury to the vascular wall in which they become attached.

The main design problem with existing IVC filters is that all the current filter designs have some component that opposes the wall of the vessel. This is either by “side struts” 200 (See FIG. 2) or by “limbs” 206 that radiate outward. These struts or limbs make-up the filter's framework and anchor the filter within a specific vascular region. Both (side struts and limbs) have edges or sharp projections that penetrate the vessel wall to prevent filter migration within the vein to undesirable locations such as the heart.

The problem regarding current filter removal is due to the struts or limbs embedding and adhering to the vascular wall. The embedding and adherence is effectuated by the formation of scar tissue between the filter components (side-struts or limbs) and the tissue of the vascular wall. In order for the IVC filter to have enough grip within the vessel wall and prevent filter displacement, a significant part if the filter must directly oppose and partially penetrate the vascular wall. Over time, scar tissue will envelope and securely attach to the filter components resulting in a filter that cannot be adequately removed without a substantial risk of vascular damage. The scarring in place, embedding and adherence are the reasons existing IVC filter designs are only approved for removal for a limited time which prevents a physician from attempting to remove a filter that has become permanently embedded within the vascular wall.

Previous attempts to create a filter which is adequately attached to the vascular wall yet will not scar in place have not met with success to date. As a result, there is a need in the art for a removable IVC vascular filter that has the following characteristics: provides adequate filtration, removal that can be performed after extended deployment time, facilitates placement and a filter element independent of structural stresses imparted by the vascular walls. The method and vascular filter described herein enables a physician to place and remove an IVC filter with minimal risk of vascular damage and at the same time increasing the time period by which the filter may be safely removed.

### SUMMARY OF THE INVENTION

To overcome the drawbacks of the prior art and provide additional benefits and features, a vascular filter system and method of implanting a vascular filter assembly is disclosed. In one embodiment, the vascular filter system comprises a filter housing and a filter element. Both the filter housing and the filter element are resilient in that they are designed to be flexible and fully collapsible. The filter housing may be configured with a plurality of filter holding members and a plurality of securing barbs extending outward from the filter housing. In one embodiment, each securing barb is angled outward from the filter housing and toward either end of the filter housing. In addition, in some embodiments, each filter holding member may extend towards the center of the filter housing.

The filter element may be configured with a plurality of limbs and a retrieval hook. The filter element is sized to fit within the filter housing and, in fact, the filter element is suspended within the filter housing by each limb engaging at least one filter holding member. In one embodiment, each limb may be curved. In some embodiments, each limb may extend from a narrow center section to either a first element end or a second element end of the filter element such as to form an hourglass shape. In another embodiment, each limb may extend from a first element end to a narrow second element end of the filter element such as to form an ogive shape.

In another embodiment of the vascular filter system, there may be a different filter element or a different filter housing. For example, the filter housing may comprise a plurality of longitudinal support struts connected by transverse angle braces. This embodiment may have at least one filter holding member attached to one or more of the longitudinal support struts or transverse angle braces and extend toward the center of the filter housing. This embodiment may have at least one securing barb attached to one or more of the longitudinal support struts or transverse angle braces and extend outward from the filter housing. In one or more embodiments, the longitudinal support struts and the transverse angle braces may be arrange such that they form a cylindrical shape.

Also as an example, the filter element may comprise a plurality of curved limbs and a retrieval hook, and be sized to fit within the filter housing such that it is suspended within the filter housing by each limb engaging at least one filter holding member. The filter element, similar to above, may have an ogive shape with a narrow apex distal end or and hourglass shape with a narrow center section in one or more embodiments. The retrieval hook may then extend from either the narrow apex end or the narrow center section of the filter element.

Some embodiments of the invention may utilize bioabsorbable materials. For example, the filter housing and the filter holding members may be formed from bioabsorbable material to allow these elements to be absorbed by the body over time. In these embodiments, the filter element may include at least one filter barb attached to and extending outward from one or more of the limbs. These filter barbs prevent the filter element from moving as the surrounding filter housing is bioabsorbed.

The filter housing and the filter element in combination may also be known as a filter assembly. The implantation of a filter assembly in a patient can occur in a variety of ways. In one embodiment, the vascular filter assembly is implanted by accessing a vein and inserting a deployment sheath. The deployment sheath in one or more embodiments, contains a filter assembly within it. The deployment sheath is advanced to a predetermined location such as the location deemed best suited to capture blood clots. Once the predetermined location is reached, a deployment member is advanced within the deployment sheath until the member contacts the vascular filter assembly. The filter assembly is released by retracting the deployment sheath while keeping the deployment member in the same location. Once released, the filter assembly will begin to expand within the vein. The deployment sheath and deployment member may then be removed from the vein. In one or more embodiments, an ogive shaped filter element may be oriented within the deployment sheath such that, upon release, the apex distal end of the filter element is upstream of the filter element's limbs.

The filter assembly may vary from one embodiment to another. For example, the filter element may include a plurality of resilient limbs and the filter housing may include a plurality of filter holding elements which suspend the filter element within the housing by engaging the limbs of the filter element. Notably, many varieties of filter assemblies, in addition to those described herein, may be similarly implanted in a patient.

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims.

### BRIEF DESCRIPTION OF THE DRAWINGS

The components in the figures are not necessarily to scale, emphasis instead being placed upon illustrating the principles of the invention. In the figures, like reference numerals designate corresponding parts throughout the different views.

FIG. 1 illustrates a typical filter placement within the inferior vena cava.

FIG. 2 illustrates two types of existing inferior vena cava filters

FIG. 3 illustrates a filter housing “A” and a removable filter element

FIG. 4 is an enlarged detail area that illustrates a securing barb of the filter housing.

FIG. 5 is an enlarged detail area that illustrates a typical filter holding member of the filter housing.

FIG. 6 illustrates one embodiment of the removable inferior vena cava filter.

FIG. 7 illustrates an assembled filter housing and filter element of FIG. 3.

FIG. 8 illustrates a collapsed filter housing and filter element assembly of FIG. 7.

FIG. 9 illustrates the collapsed filter housing and filter element assembly of FIG. 8 as contained within a deployment sheath.

FIGS. 10a through 10g illustrate the deployment of the filter housing and filter element assembly of FIG. 7.

FIGS. 11a through 11h illustrate the removal of the inferior vena cava filter.

FIGS. 12a and 12b illustrate an alternate embodiment which utilizes a bioabsorbable filter housing.

FIGS. 13a through 13g illustrate a “time-lapse” image series of the filter housing dissolving and corresponding changes in filter positioning.

FIG. 14 illustrates an alternate embodiment for a compact removable inferior vena cava filter design.

FIG. 15 illustrates an alternate embodiment for an inferior vena cava filter design providing enhanced hemo dynamic flow characteristics.

FIGS. 16a and 16b illustrate alternate embodiments for an inferior vena cava filter including a filter housing individually and combined in both an expanded and collapsed state.

FIG. 17 is an enlarged detail area that illustrates a typical filter holding member of the filter housing in accordance with an alternate embodiment of the inferior vena cava filter design.

FIGS. 18a and 18b are an enlarged detail area that illustrates an alternate embodiment of the filter holding member.

FIGS. 19a and 19b illustrate another alternate embodiment for the filter holding member.

FIG. 20 illustrates another embodiment filter and housing in various states.

FIG. 21 illustrates yet another embodiment filter and housing.

### DETAILED DESCRIPTION OF THE PREFERRED EMBODIMENTS

In the following description, numerous specific details are set forth in order to provide a more thorough description of the present invention. It will be apparent, however, to one skilled in the art, that the present invention may be practiced without these specific details. In other instances, well-known features have not been described in detail so as not to obscure the invention.

One of the primary concerns regarding deep vein thrombosis (DVT) is that should the thrombus (blood clot) dislodge from the origination location, the thrombus may travel to another region of the circulatory system and cause injury and or death to the subject. For example, if a DVT dislodges it may migrate through the heart and eventually re-lodge in the lung of the subject thus causing a Pulmonary Embolism which prevents adequate circulation and can cause sudden death of the subject. By placing an intravenous filter in the inferior vena cava, the blood clot may be captured and prevented from migrating to vulnerable regions of the circulatory system. The filter may be placed in other veins or at other locations such that the filter is positioned to capture a thrombus prior causing damage or medical complications to the patient.

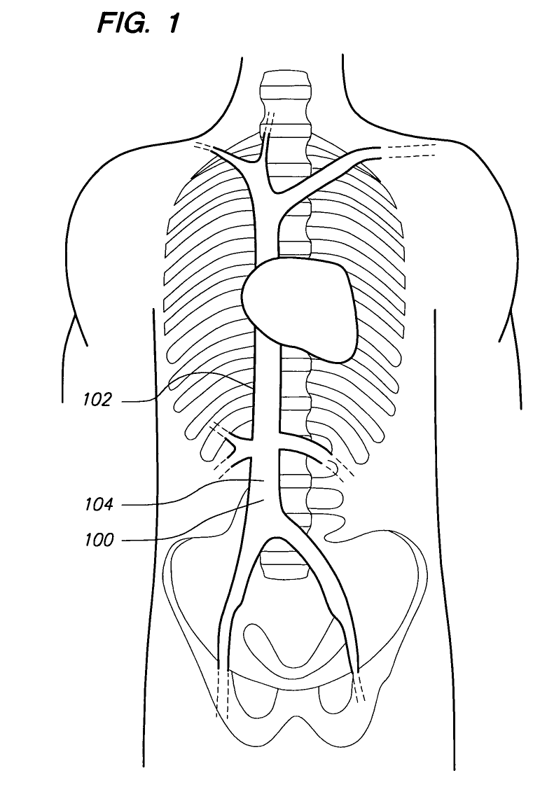

Referring now to the drawings, FIG. 1 illustrates the typical location 100 for surgically implanting an inferior vena cava filter (IVC filter) using a large bore vein such as the inferior vena cava 102 located in the patient's upper abdomen. The IVC filter is typically deployed within the large bore vein using a large bore catheter and traditional access through a larger vein such as the patient's common femoral vein, the veins of the upper arm or the internal jugular vein. Placement of the IVC filter 100 is generally located within the inferior vena cava 102 and below the renal veins 104 as annotated in FIG. 1.

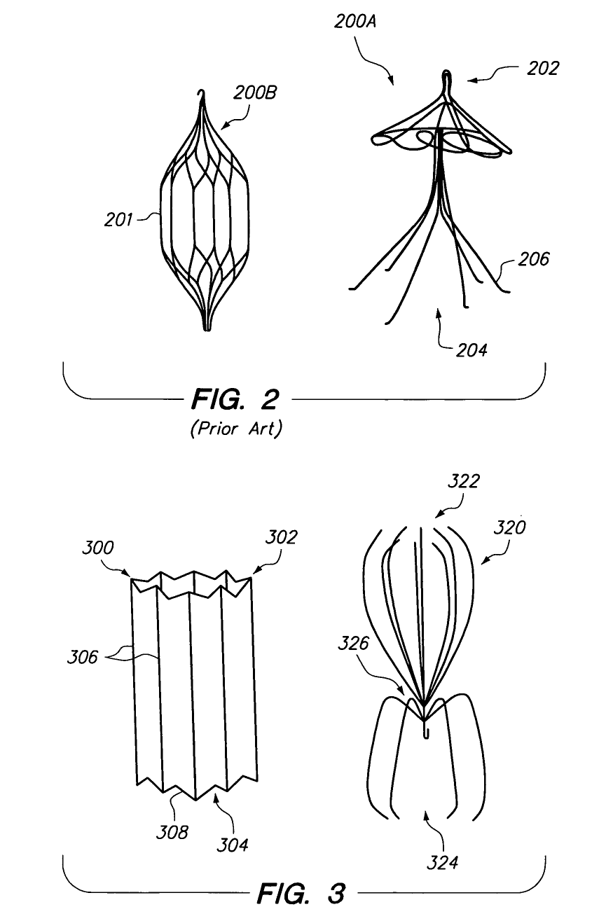

FIG. 2 illustrates two types of existing inferior vena cava vascular filters that are surgically implanted into a patient. The IVC filter 200 is commonly deployed using a large bore catheter and access to a large bore vein such as the inferior vena cava. The typical IVC filter 200A has a first end 202 and a second end 204 where the second end comprises a plurality of individual wire components 206 or limbs that are in contact with the vascular walls. In another version of the typical IVC filter 200B the filter is generally cylindrical in shape and has a plurality of side-strut edges 201 that engage the inner vascular walls.

FIG. 3 illustrates a filter housing 300 and a removable filter element 320 as one embodiment of the present invention. The filter housing 300 comprises a first housing end 302 and a second housing end 304 and the housing is generally cylindrical in shape, a plurality of longitudinal support struts 306 and a plurality of transverse angle braces 308 in which both the struts and the braces provide structural stability for the filter housing. The transverse angle braces 308 are formed around the circumferential edges of each the first and second ends 302/304 respectively. A filter element 320 comprises a first element end 322 and a second element end 324 and is generally hour-glass in shape with a narrowing center section 326. The filter element 320 is sized for insertion within the filter housing 300. In one embodiment both the filter housing 300 and filter element 320 are designed to be flexible, resilient and fully collapsible so that they may be advanced, as a single assembly, into a vascular region using a catheter sheath. Note that the term resilient, in one or more embodiments, may mean that the filter housing 300 and the filter element 320 are flexible and may fully or partially collapse and substantially or completely recover their original shape. The steps involved in deployment and removal are discussed in greater detail below. The filter housing 300 and filter element 320 are contemplated to be fabricated from a material suitable for implantation within a biological subject. Some examples of suitable materials are titanium, polycarbonate, polypropylene or other hypoallergenic materials that provide adequate spring tension, form-factor/shape memory and compatibility with living tissue.

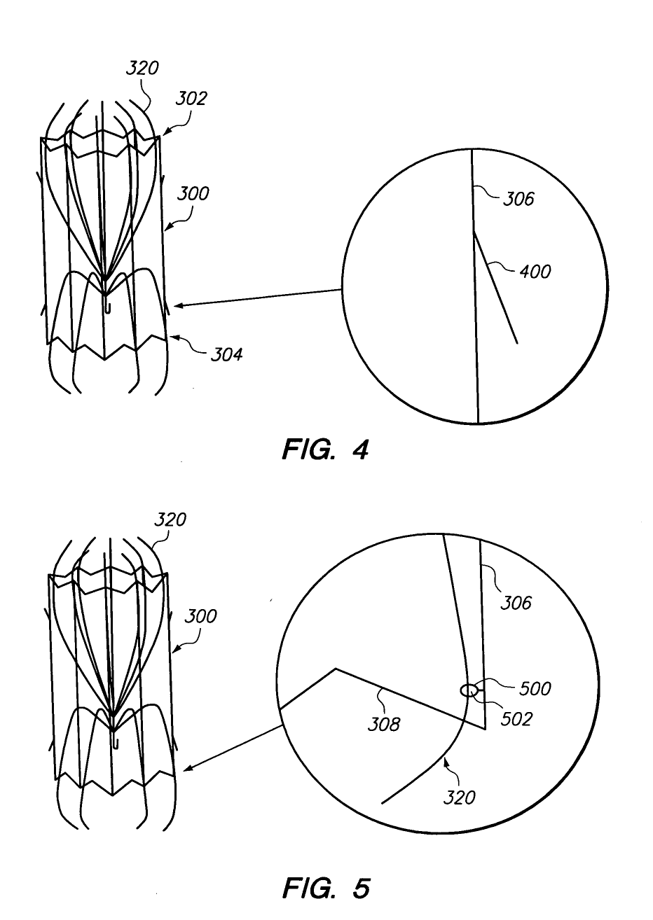

Reference is now made to FIG. 4 which provides an enlarged detail area illustrating a securing barb 400 of the filter housing 300. The securing barb 400 extends radially outward from the filter housing 300 and is angled towards an end of the housing. In one embodiment, there is a plurality of securing barbs 400 around the circumference of the filter housing 300. It is further contemplated that each end of the filter housing is fitted with the securing barbs 400 such that the barbs resist and prevent movement of the filter housing 300 with respect to the inner surface of the vascular wall.

Additionally, there may be other arrangements of the securing barbs 400 such as, but not limited to, a centrally located series of barbs. The securing barbs 400 are arranged in an opposing geometric orientation. For example, in FIG. 4 a first housing end 302 has a securing barb 400 that is generally orientated in an upward direction. The second housing end 304 has a securing barb 400 that is orientated in downward/opposite direction. As a result, once the securing barbs 400 penetrate and engage the vascular wall, the filter housing 300 will remain in place and cannot move and/or translate within the vein. It is further contemplated that the securing barbs 400 are integrally formed with the longitudinal support struts 306 of the filter housing 300. The securing barb 400 may have other geometric shapes such as an inclined plane, a radial boss or other protrusion that is extends away from the filter housing and embeds into the vascular wall.

FIG. 5 provides an enlarged detail area that illustrates a filter holding member 500 of the filter housing 300. The filter holding member 500 protrudes generally from the longitudinal support strut 306 toward the center of the filter housing 300. The filter holding member 500 is contemplated to be integrally formed with the longitudinal support strut 306. A plurality of filter holding members 500 is preferably located at each end of the filter housing 300 and distributed around the circumference of the housing. The filter holding members 500 are configured to receive at least one of the filter limbs through an aperture 502 formed within the member. The filter limbs project through the aperture 502 at multiple locations along both the superior and inferior leading edges of the filter housing 300. The filter holding member 500 suspends, supports and spaces the filter limbs away from the inner vascular walls. By spacing the filter limbs away from the inner vascular wall, the filter element 320 will not contact, scar-in or otherwise adhere to the vascular wall.

In one embodiment, a filter holding member 500 is provided for each filter limb of the filter element 320. It is contemplated that the filter holding members 500 may be constructed in other geometric shapes and configurations such as a protruding boss, flange, post or support. In any of these configurations, an important aspect of the filter holding member 500 is to provide a member that releasably retains a filter limb while at the same time constraining the filter limb such that the limb will not contact the inner vascular wall.

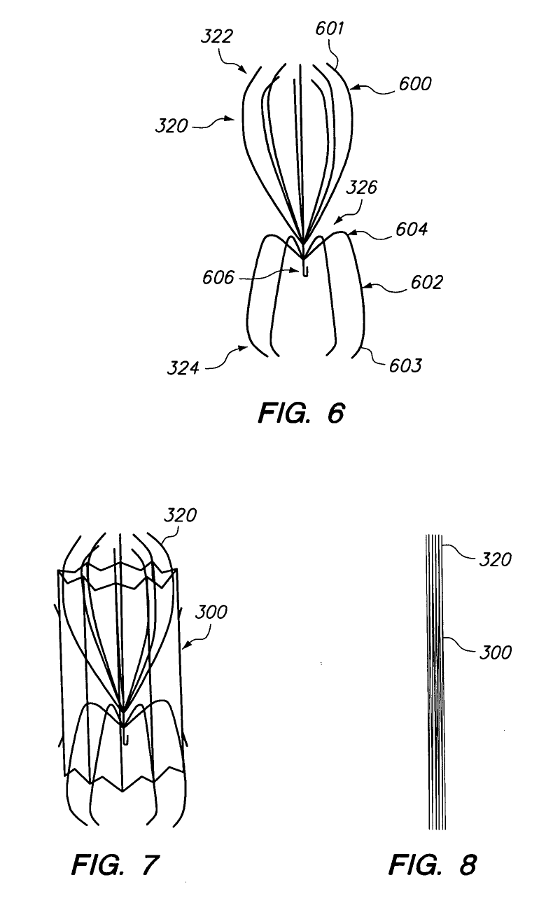

FIG. 6 illustrates one embodiment of the removable IVC filter and specifically illustrates a filter element 320. The filter element 320 comprises a first element end 322 and a second element end 324 and is generally hour-glass in shape with a narrowing center section 326. The filter element 320 is sized for insertion within the filter housing 300. The filter element 320 has a plurality of limbs that extend from the center section 326 toward the ends. The limbs are fabricated such that they are flexible to the extent necessary to deform and straighten during the deployment and removal procedure. As illustrated in FIG. 6, a plurality of upper limbs 600 extend in a curved fashion from the center section 326 towards a first element end 322. The distal end of the upper limbs 600 is configured with a smooth upper curve 601 that is directed internally and towards the center of the filter element 320.

A plurality of lower limbs 602 extend from the center section 326 towards a second element end 324. The lower limbs 602 are configured with an intermediate curve 604 that initially curves towards the first element end 322 and subsequently recurves towards the second element end 324. The intermediate curve facilitates the collapsibility of the filter element 320 during the removal process. The distal end of the lower limbs 602 is configured with a smooth lower curve 603 that is directed internally and towards the center of the filter element 320. In one embodiment, there are 8 upper limbs 600 and 4 lower limbs 602 that extend from the center section; however, it is contemplated that more or less limbs or any combination of upper or lower limbs may be used.

Additionally, FIG. 6 illustrates a retrieval hook 606 that is integrally formed from the center section 326 and extends towards the second element end 324. The retrieval hook 606 facilitates the removal process by providing a centrally located grasping point on the filter element 320 by which a snaring loop may be attached and the filter element subsequently drawn into a removal catheter. A detailed disclosure of the removal process is provided below.

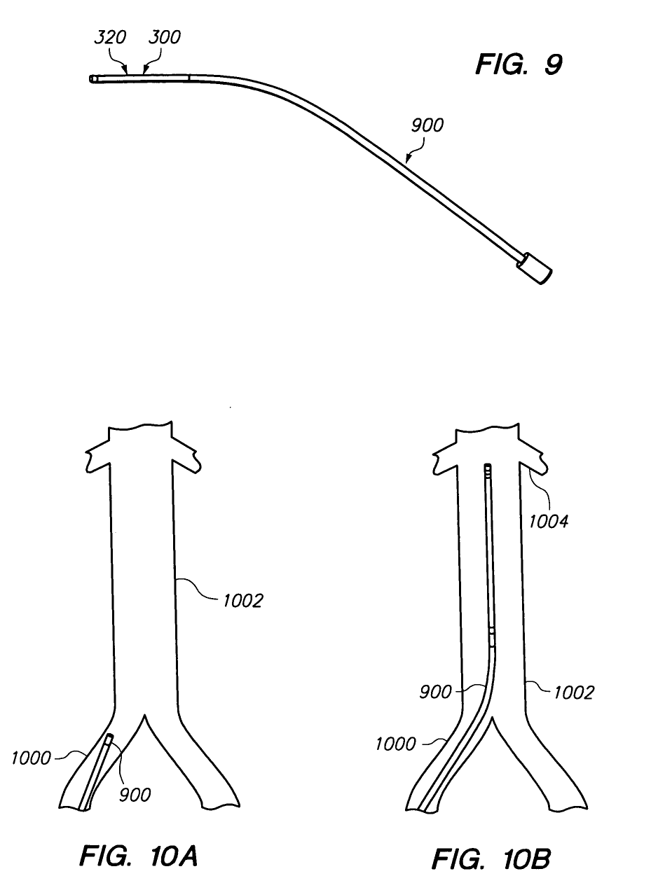

Turning now to FIGS. 7 through 9, which illustrate an assembled IVC filter 700 of FIG. 3 in both a deployment and a collapsed state. Additionally, in FIG. 9, the collapsed IVC filter assembly of FIG. 8 is illustrated as contained within a deployment sheath 900. The IVC filter 700 assembly of FIG. 7 comprises a filter housing 300 and a filter element 320. In FIG. 8, the IVC filter assembly is collapsed and occupies a substantially smaller volumetric region. The collapsed IVC filter assembly is then placed within a deployment sheath 900 as illustrated in FIG. 9. The deployment sheath 900 and IVC filter is generally preloaded by the manufacturer.

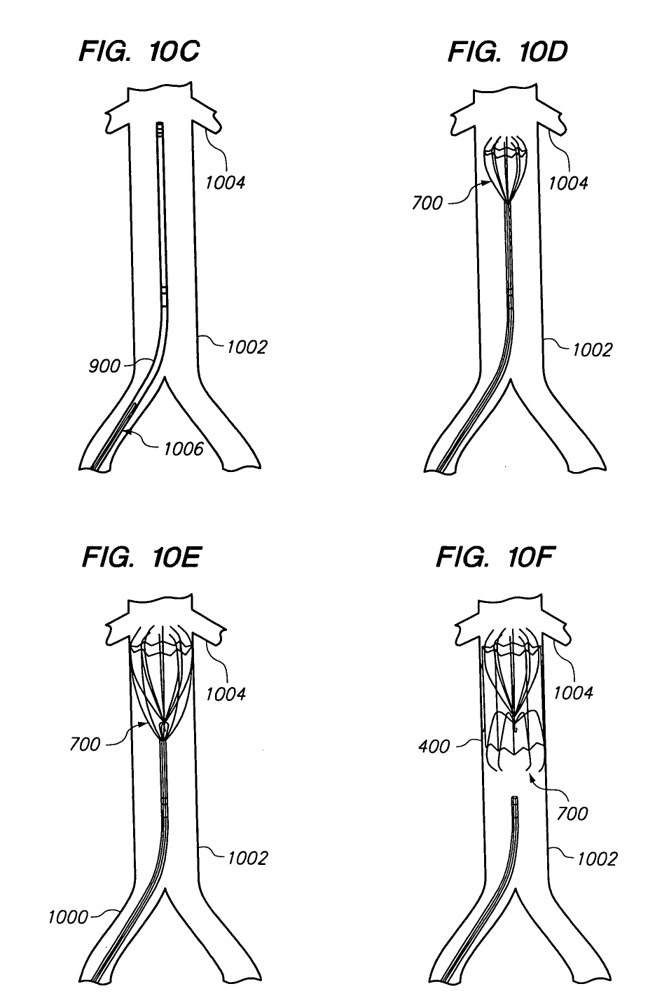

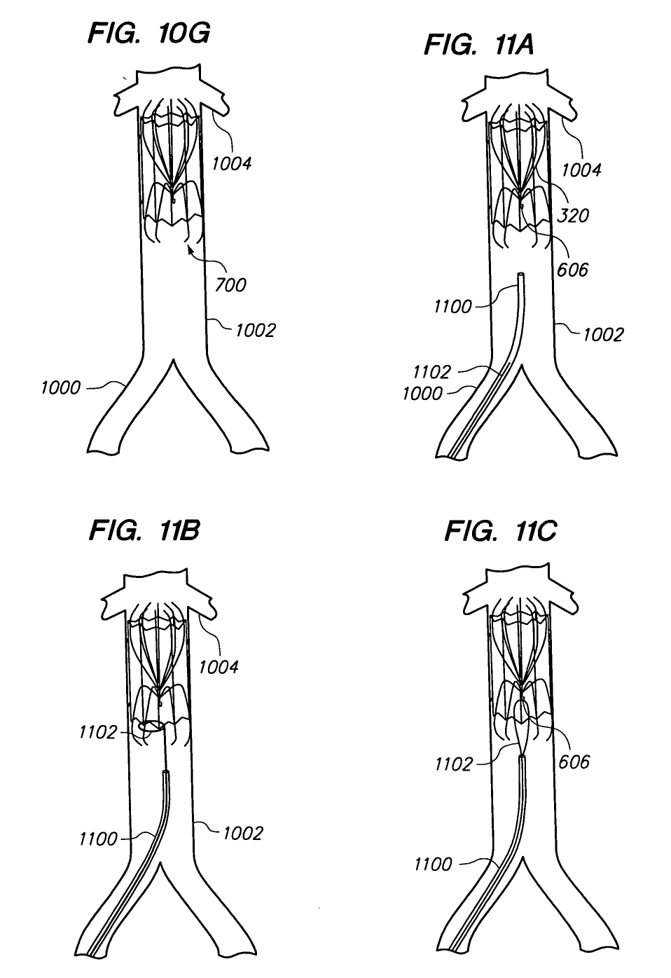

The following disclosure is directed to one implementation for deployment of the IVC filter described herein. Reference is now made to FIGS. 10a through 10g individually and in combination for illustrating the deployment of the IVC filter assembly 700. The IVC filter assembly 700 is deployed into the inferior vena cava in a similar fashion as most of the current IVC filters. Access is performed using standard techniques into a patient's vein. The veins that are commonly used include the large veins of the groin, such as the common femoral vein 1000, the larger veins of the upper arm or the large vein in the neck—the internal jugular vein. Once access is obtained, a guiding wire is advanced into the inferior vena cava. Over this wire (not shown for clarity) the deployment sheath 900, which is preloaded the IVC filter assembly 700, is advanced in to the inferior vena cava 1002, See FIG. 10a. Current practice uses contrast (radiographic dye) injected to provide visual navigation and mapping of the inferior vena cava during the procedure.

The deployment sheath 900 is then advanced to the appropriate position within the inferior vena cava 1002 which is generally below the inflow from the renal veins 1004, see FIG. 10b. Next, in FIG. 10c, a deployment member or pusher 1006 or is then advanced within the deployment sheath 900 to the base of the collapsed IVC filter assembly 700. The IVC filter assembly 700 is then slowly deployed by holding the pusher 1006 in a fixed position and pulling the outer deployment sheath 900 back, see FIG. 10d. This technique then slowly exposes the collapsed filter while maintaining the filter's position relative to the inferior vena cava 1002. The prior form-factor/shape memory and internal tension of the IVC filter assembly 700 causes it to self-expand and “open” within the inferior vena cava 1002, see FIG. 10e.

As the IVC filter 700 expands, the filter housing meets the inner wall on the inferior vena cava and the securing barbs 400 slightly penetrate and engage the inner wall, see FIG. 10f. Also illustrated in FIG. 10f, the filter housing 300 and the filter element 320 expand simultaneously to complete the deployment process. Upon complete self-expansion of the IVC filter assembly 700, the deployment sheath 900 and the pusher 1006 are withdrawn from the insertion site and the patient's vascular system, see FIG. 10g.

It is further contemplated that the deployment of the IVC filter assembly may be performed in other vascular regions to prevent thrombus migration. Correspondingly, the removable filter disclosed herein may be deployed within other regions of a patient's body as required by the specific medical requirements or case stratagem.

The need to remove a filter arises when a patient is no longer at risk for clot formation and the possibility of clot migration and pulmonary embolism has subsided. There are complications that can occur when a filter is left in place such as scarring of the inferior vena cava and possible metal fatigue/fracture of the filter. In addition, blood flow is hindered or restricted when the filter remains in place. Currently it is desirable to remove filters when they are no longer necessary for the patient's health. However, the currently available IVC filters typically remain with the patient for life because there is a small time period in which the filter can be safely removed; outside of this time period there is substantial risk of vascular damage to the patient if filter removal is performed. The present invention provides an IVC filter that can remain deployed within a patient for a significant time period while at the same time is removable throughout this period.

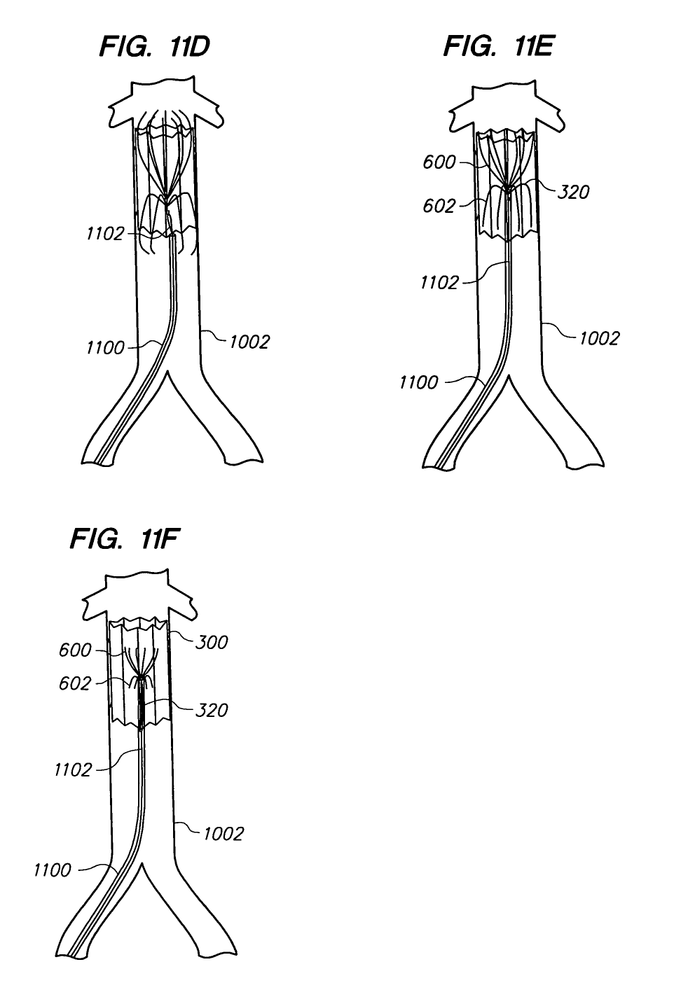

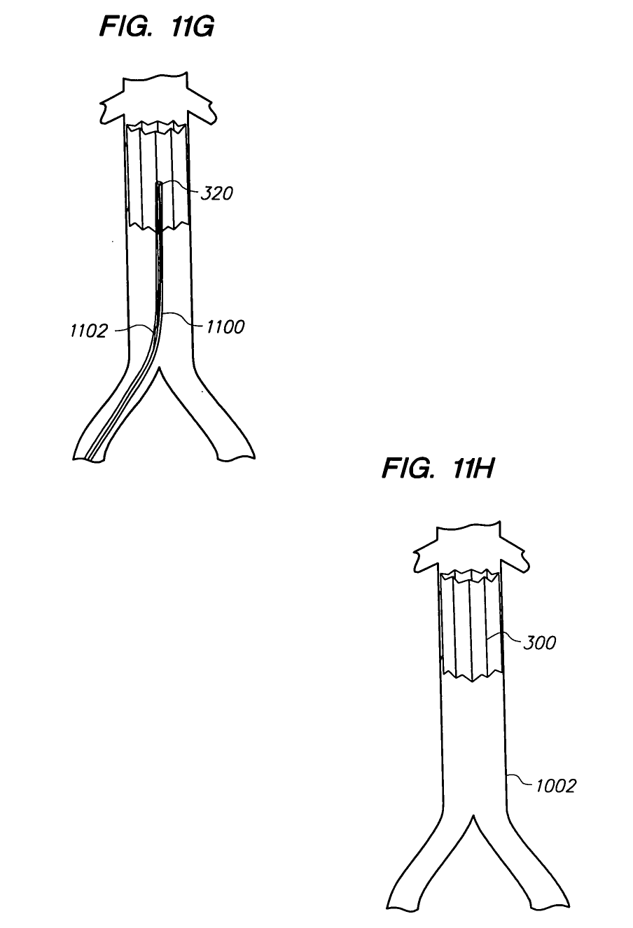

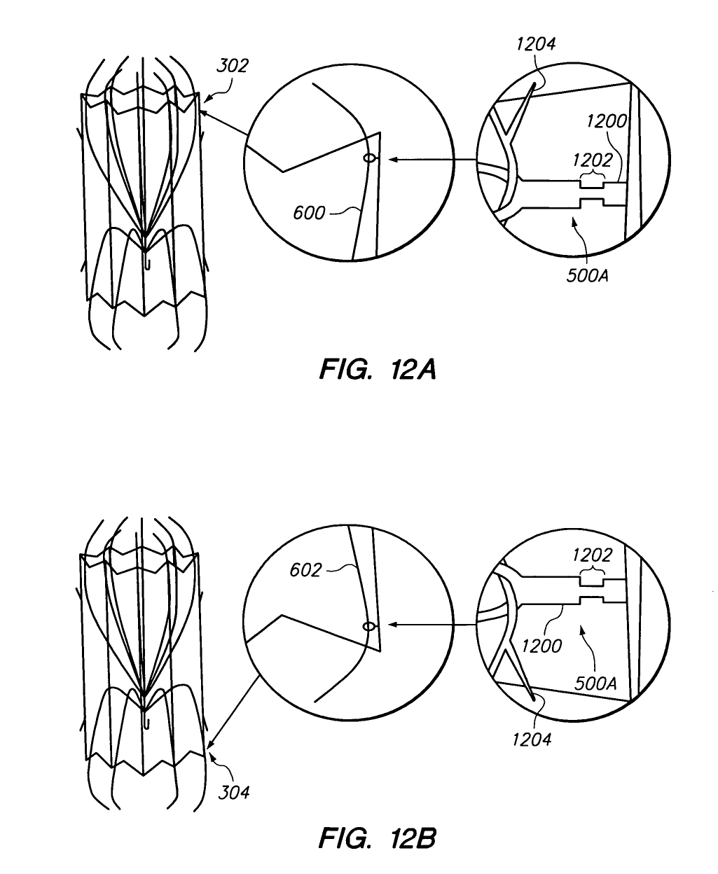

Reference is now made to FIGS. 11a through 11h individually and in combination for illustrating the removal of the filter element 320 from the IVC filter assembly 700. In FIG. 11a, a snaring catheter 1100 and snare wire 1102 is advanced to the location of the IVC filter assembly 700 through access performed using standard techniques into a patient's vein. The veins that are commonly used include the large veins of the groin, such as the common femoral vein 1000, the larger veins of the upper arm or the large vein in the neck—the internal jugular vein. The snaring catheter 1100 is advanced until within in close proximate location with the retrieval hook 606 of the filter element 320 (See FIG. 6). Once the snaring catheter 1100 is in place, the snare wire 1102 is advanced through and beyond the end of the snaring catheter as shown in FIG. 11b. The snaring wire 1102 is then advanced and manipulated until the snaring wire engages the retrieval hook 606, see FIG. 11c.

Next, the snaring catheter 1100 is advanced along the snaring wire 1102 until the catheter is proximate to the retrieval hook 606 as illustrated in FIGS. 11d and 11e. Once the snaring catheter 1100 contacts the retrieval hook 606, the snaring wire 1102 is retracted through the catheter and the filter element 320 is drawn into the snaring catheter, see FIG. 11e. As the filter element 320 is drawn into the snaring catheter 1100, the filter limbs (both upper and lower) will deflect and slide through their respective apertures 502 of filter holding members 500 (See, FIG. 5). The lower limbs 602 of the filter element 320 are deflected/folded in an upward direction and into the catheter while at the same time the upper limbs 600 deflect/collapse and are drawn into the catheter.

In practice, the snaring catheter 1100 is slightly advanced in unison while the snaring wire 1102 is retracted. The combination of advancing the snaring catheter 1100 while retracting the snaring wire 1102 is considered a standard snaring technique in the intravascular medical field. In FIG. 11f, the filter element 320 is further drawn into the snaring catheter 1100 as the filter housing 300 remains in place. The filter element 320 is continually drawn into the snaring catheter 1100 by retracting the snaring wire 1102 until the filter element is completely within the snaring catheter, as shown in FIG. 11g. Once the filter element 320 is within the snaring catheter 1100, the catheter with the filter element 320 contained therein is removed from the patient, the filter housing 300 may remain behind and the removal procedure is complete, see FIG. 11h. Alternatively, the housing 300 could be removed.

In one embodiment, the filter housing and filter holding members are fabricated from a bioabsorbable material. As a result of the bioabsorbable material properties, the filter housing and holding members will degrade overtime resulting in a retrievability time span that is determined by the bioabsorbable material properties (various time spans can be developed using the properties of the bioabsorbable materials). The IVC filter fabricated from bioabsorbable material may have substantially similar structure and is deployed in a similar fashion as previously described. Once deployed in the patient, the filter will function substantially in a similar fashion filtering the blood of any migrating thrombus. Over time the bioabsorbable materials will dissolve, the filter holding members will separate from the filter housing and the filter element limbs (fitted with their own securing barbs) will attach to the inner vascular wall. Then overtime the remaining filter housing structure will dissolve permanently leaving only the filter element in place.

Reference is now made to FIGS. 12a and 12b, which illustrate an enlargement of the first housing end 302 (FIG. 12a) and a second housing end 304 (FIG. 12b) of the filter housing. In this embodiment, there are two primary structural differences in the filter housing and the filter element. The filter housing has a plurality of filter holding members 500A that are integrally formed with the filter housing using the bioabsorbable material. The filter holding member 500A has a base 1200 configured with a narrowing section 1202. The narrowing section 1202 of the base 1200 is designed such that section 1202 will bioabsorb prior to the structure of the filter housing. Once the narrowing section 1202 has been absorbed the filter holding member 500A will separate from the filter housing. Upon separation of the filter holding member 500A from the filter housing, the limbs 600/602 (see FIG. 6) of the filter element are now free to deflect radially outward and engage the inner surface of the vascular walls. In this embodiment, it is contemplated that the filter limbs 600/602 are configured with a plurality of filter barbs 1204 similar to the securing barbs 400 (FIG. 4) of the filter housing. The primary function of the filter barbs 1204 is to retain the filter element in place subsequent to the absorption of the filter housing and filter holding members. Correspondingly, once the filter element has engaged the inner vascular wall the filter element will begin to adhere to the vascular wall and will eventually become permanently attached within the vessel (due to scar tissue growth around the filter).

In operation, the bioabsorbable filter housing generally functions the same as the previously described filter housing with the exception that after a certain amount of time the housing will degrade and the filter element will become permanent within the patient. For example, in one embodiment the bioabsorbable material of the filter housing may begin to breakdown after 9 months. This allows the filter element to be completely removable for up to 9 months using the removal process previously described. If the filter element is removed within this time span, there will be no remnants of the IVC filter because the filter housing will eventually bioabsorb overtime. In contrast, the non-bioabsorbable filter housing will always remain with the patient even after the filter element has been removed. After 9 months the filter housing and filter holding members begin to bioabsorb. It is preferred that the first component to fully absorb is “the base” of the filter holding member which spaces the filter element limbs away from the interior vascular wall as described above with reference to FIG. 5. The pre-formed limbs of the filter element are designed to “spring” outward and hook their “barbs” into the interior vascular wall subsequent to the separation of the filter holding members from the filter housing. Additionally, the filter housing continues to bioabsorb until only the filter element remains. The filter element will permanently adhere to the vessel over the next few months. It is further contemplated that other time intervals may be developed depending on the bioabsorbable material properties and structure of the filter holding members 500A. For example, the filter holding members 500A may be configured as boss or flange that absorbs at various rates resulting in alternate time intervals between initial deployment and engagement of the filter element limbs with the vascular wall.

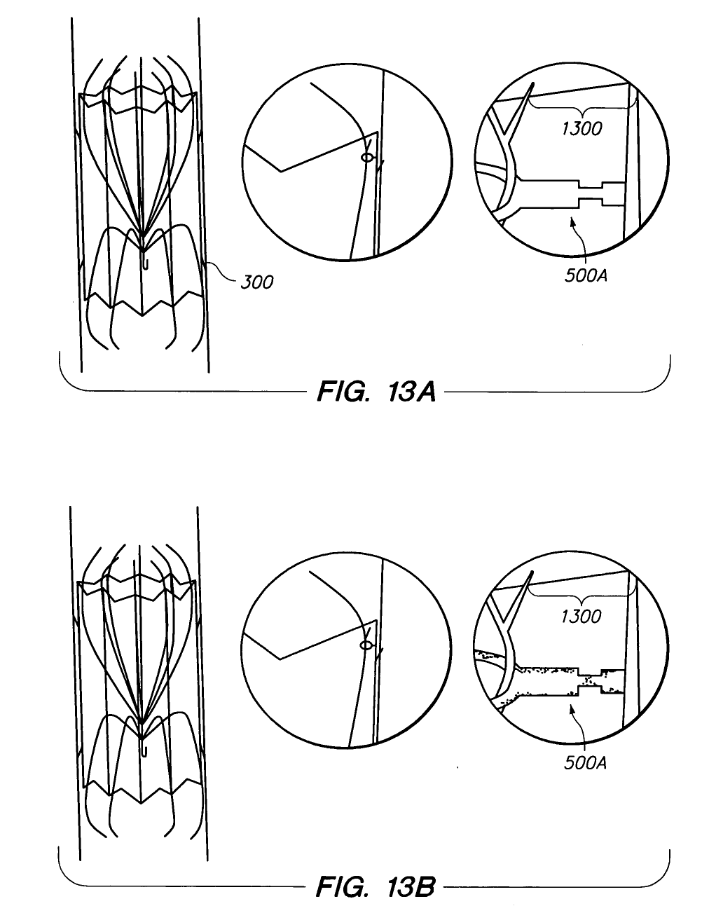

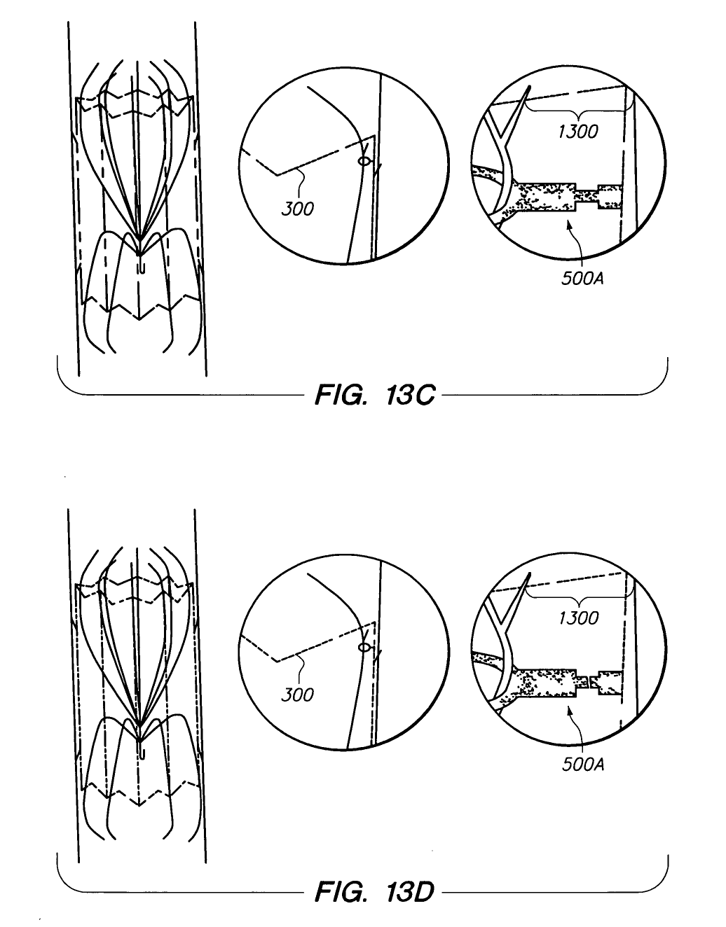

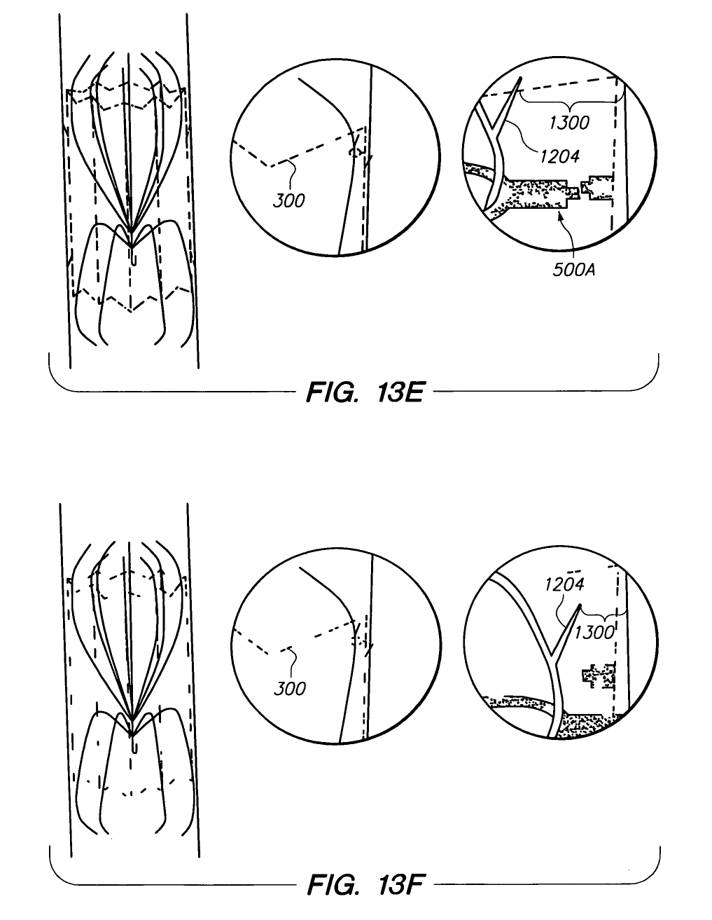

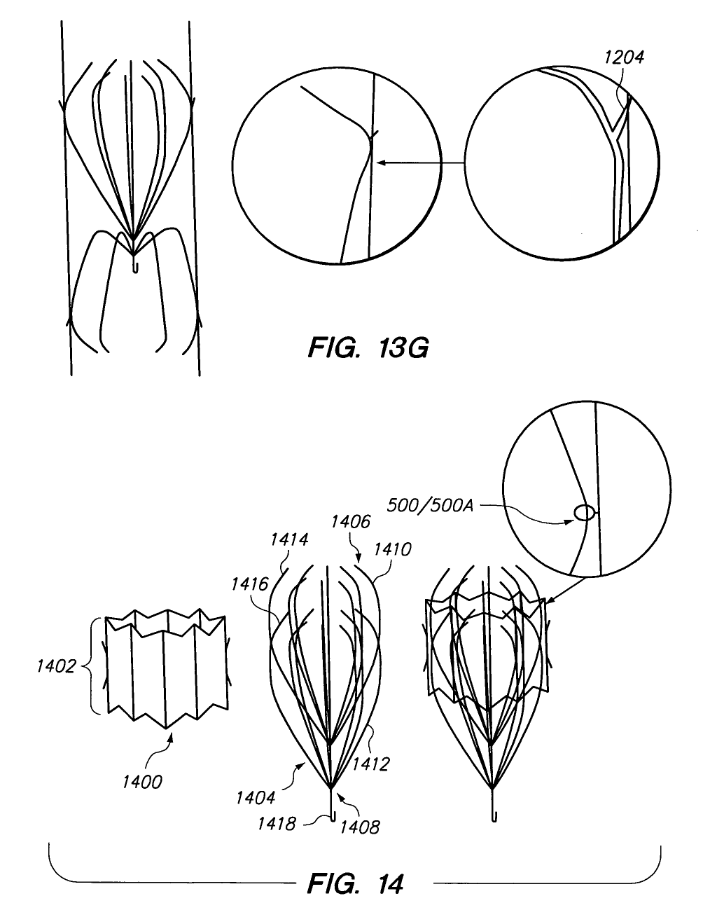

Reference is now made to FIGS. 13a through 13g individually and in combination which illustrate a “Time-Lapse” absorption of the filter housing 300 and filter holding member 500A. FIG. 13a shows the IVC filter as initially deployed and prior to any absorption of the filter housing 300 or filter holding member 500A. The filter holding member 500A is intact and has not begun to degrade. Additionally, the filter limb and filter barb 1204 are spaced 1300 apart from the vascular wall. In FIGS. 13b through 13d the filter housing and filter holding member begin to absorb and become structurally weaker. In FIG. 13e, the filter holding member 500A has separated from the filter housing due to the narrowing section being absorbed to the point of structural failure. At this point the filter element limb deflects radially outward due to internal forces/spring tension of the limbs. As a result, the space 1300 between the filter limb and the vascular wall is substantially reduced permitting the filter limb and filter barb 1204 to engage the inner surface of the vascular wall as shown in FIGS. 13f and 13g.

FIG. 14 illustrates another embodiment of the removable IVC filter and specifically illustrates a filter housing 1400 that is substantially smaller than the previously described housing. The filter housing 1400 is essentially similar in construction and configuration as the previous embodiments with the exception of the longitudinal dimension 1402. It is contemplated that this embodiment may use a filter housing 1400 fabricated from either a bioabsorbable or non-bioabsorbable material. The longitudinal dimension 1402 is reduced due to the modified configuration of the filter element 1404.

As illustrated in FIG. 14, the filter element 1404 comprises a first element end 1406 and a second element end 1408 and is generally ogive in shape. The filter element 1404 is sized for insertion within the filter housing 1400. The filter element 1404 has a plurality of limbs that extend from the second element end 1408 toward the first element end 1406. The limbs are fabricated such that they are flexible to the extent necessary to deform and deflect during the deployment and removal procedure. As illustrated in FIG. 14, a plurality of upper limbs 1410 extend in a curved fashion from the second element end 1408 towards a first element end 1406. The distal end of the upper limbs 1410 are configured with a smooth upper curve 1414 that is directed internally and towards the center of the filter element 1404.

A plurality of lower limbs 1412 extend from the second element end 1408 toward the first element end 1406. The distal end of the lower limbs 1412 are configured with a smooth lower curve 1416 that is directed internally and towards the center of the filter element 1404. In one embodiment, there are 8 upper limbs 1410 and 8 lower limbs 1412 that extend from the from the second element end 1408; however, it is contemplated that more or less limbs or any combination of upper or lower limbs may be used.

Additionally, FIG. 14 illustrates a retrieval hook 1418 that is integrally formed from the second element end 1408. The retrieval hook 1418 facilitates the removal process by providing a centrally located grasping point on the filter element 1404 by which a snaring loop may be attached and the filter element subsequently drawn into a removal catheter.

It is contemplated that the filter element shown in FIG. 14 may be fitted with a plurality of filter barbs integrally formed with the limbs 1410/1412 for use when implemented with a bioabsorbable filter housing. The function of the filter element 1404 with the bioabsorbable filter housing is essentially similar to the previously described embodiment of FIG. 12.

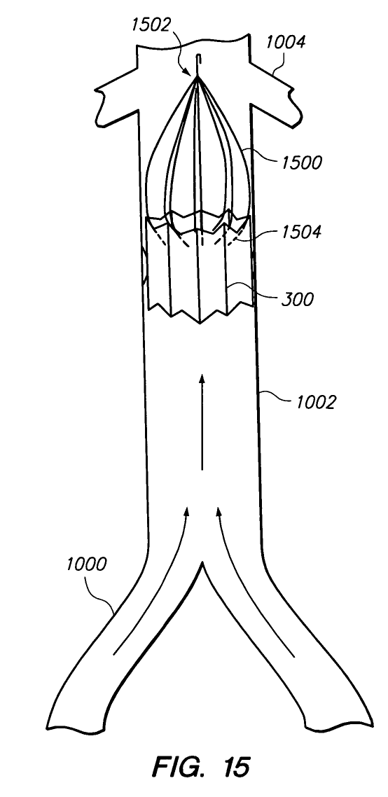

Another IVC vascular filter embodiment and deployment configuration is illustrated in FIG. 15. As shown, there is a filter housing 300 which is essentially the same as disclosed previously. However, an alternate filter element 1500 is combined with the filter housing 300. In this deployment configuration, the filter element 1500 is arranged such that an apex distal end 1502 is located upstream from the ends of the filter element limbs 1504. As a result, the hemodynamic flow through the IVC filter is enhanced, particularly when a thrombus is captured in the filter element. In FIG. 15, the circulatory flow is illustrated as it proceeds from the common femoral veins 1000 which merge with the inferior vena cava 1002, through the inferior vena cava and then through the IVC filter. Studies have been conducted which analyze the fluid dynamics of a deployed IVC filter and have shown that benefits are realized by placing the filter element 1500 in the orientation illustrated in FIG. 15. In other embodiments other placement of the IVC filter may be desired and changes to the orientation do not preclude coverage by the claims which follow.

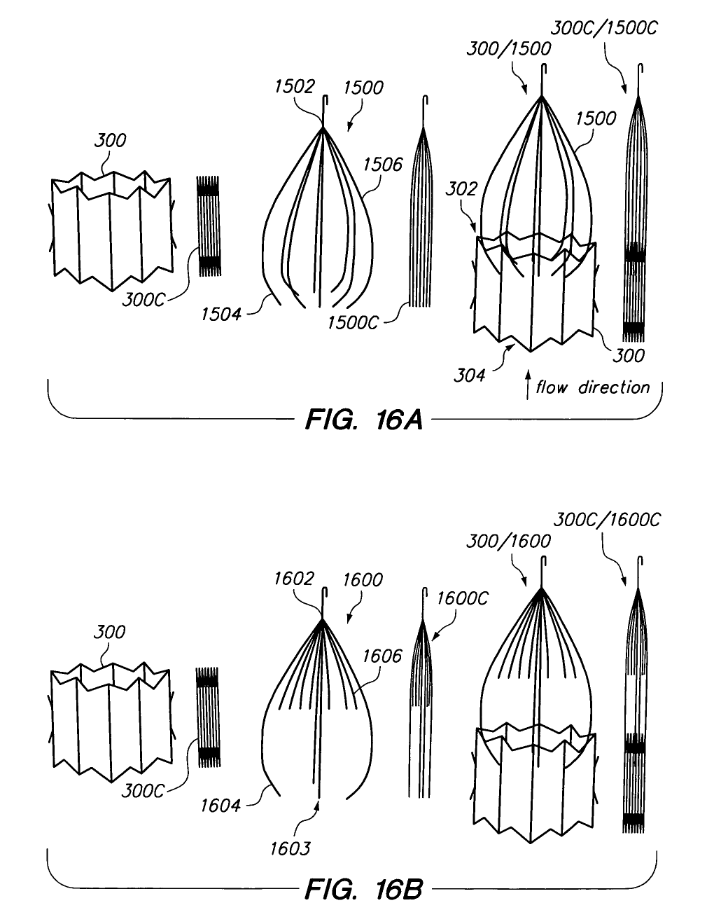

In FIG. 16a, a series of illustrations show a filter housing 300 and another embodiment of the filter element 1500. The filter housing 300 and filter element 1500 are shown in a collapsed state and are designated 300c and 1500c respectively. Additionally, FIG. 16a illustrates one variation of an assembled IVC filter 300/1500 comprising the filter housing 300 and filter element 1500 and is also shown in a collapsed stated designated 300c/1500c. In the view of the IVC filter assembly 300/1500, the filter element substantially extends away from a first housing end 302 and generally away from a second housing end 304. The apex distal end 1502 and primary ogive shape are oriented in the direction of circulatory flow. The filter element 1500 comprises a plurality of filter limbs 1506 originating from the apex distal end 1502, curving away from the distal end and into the direction of vascular flow.

Another embodiment of an IVC vascular filter is illustrated in FIG. 16b. In similar respect to FIG. 16a, the filter housing 300 and filter element 1600 are shown in an expanded and collapsed state. The collapsed illustrations are designated by reference numerals ending in “c” for example the expanded filter housing is designated 300 while the collapsed filter housing is designated 300c. In this new embodiment, the filter element 1600 comprises a plurality of primary attachment limbs 1604 which are utilized to secure and retain the filter element within the filter housing 300. As previously described, the filter limbs and the attachment limbs 1604 originate at the apex distal end 1602 and extend in curved trajectories towards a proximate end 1603 of the filter element 1600.

In one embodiment of filter element 1600, there are only a few primary attachment limbs 1604 provided for attachment of the filter element to the filter housing 300. For example, one embodiment may only provide 4 attachment limbs 1604. However, it is contemplated that other combinations or number of attachment limbs 1604 may be implemented as required. By reducing the number of primary attachment limbs 1604, the IVC vascular filter assembly can be fabricated to fit within a smaller catheter because there are less limbs directly attached to the filter housing 300 and correspondingly occupy less volume.

The filter element 1600 further comprises a plurality of intermediate filtering limbs 1606 which provide filtering means for the regions between the primary attachment limbs 1604. As a result, the filtering limbs 1606 are not connected or attached to the filter housing 300 and generally extend from the apex distal end 1602 towards the proximate end 1603. The filtering limbs 1606 may be either curved, straight or a combination of geometric transformations (such as spiral, vortex, etc) extending from the apex distal end 1602. An important aspect of the filtering limbs 1606 is that they are not attached to the filter housing 300 but rather occupy the volumetric region between the primary attachment limbs 1604 and provide a means for filtering/capturing thrombi flowing through the IVC filter.

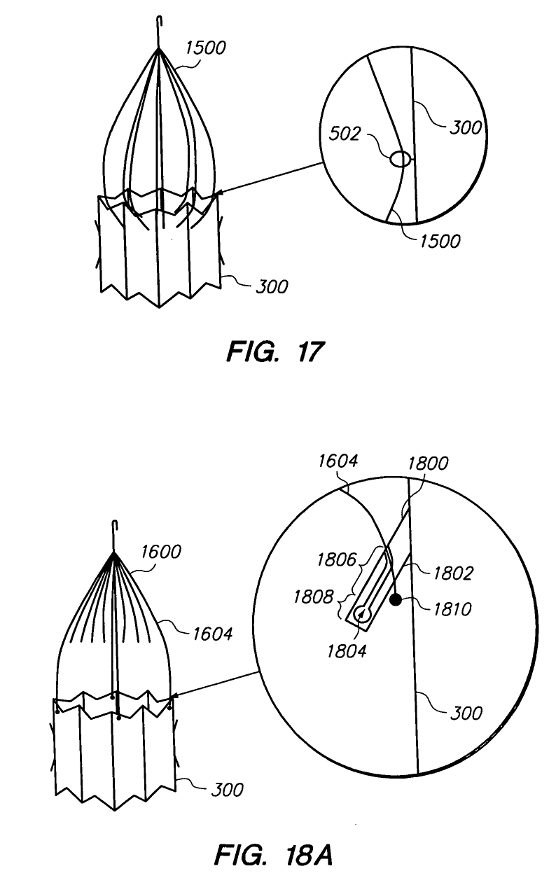

Reference in now made to FIG. 17 which illustrates a filter holding member 502 of the filter housing 300 and an alternate embodiment of the IVC filter design. In this embodiment, the filter element 1500 is retained in the filter housing 300 by way of filter holding members 502 as previously discussed with reference to FIGS. 5 and 12. Generally, the limbs of the filter element 1500 are releasably retained within the filter holding members 502.

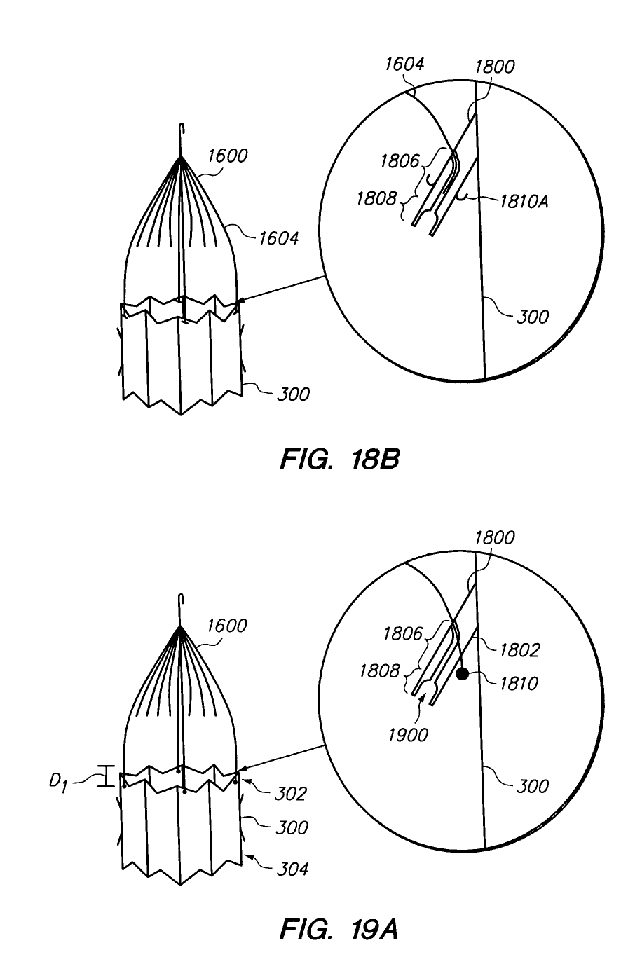

Another embodiment is shown with reference to FIG. 18a which illustrates an alternative filter holding member 1800. The filter holding member 1800 has a base 1802 that is integrally formed from the filter housing 300 and extends towards the center of the housing. The base 1802 is configured with a keyway 1804 which releasably retains a portion of the filter limb 1604. The keyway 1804 has a narrow slot 1806 which is sized to accommodate the minimal dimensions (diameter) of the filter limb 1604 therein. The keyway 1804 is further configured with an aperture 1808 or opening which is sized to allow the end of the filter limb 1604 to pass there through.

In one embodiment, the filter limb 1604 has a retention end member 1810 such as a bead, ball, or other geometric configuration that is larger in dimension (diameter) than the rest of the filter limb 1604. The retention end member 1810 is sized to pass through the aperture 1808 and thereby permit the shaft, shank or length of the filter limb 1604 to engage the narrow slot 1806 of the filter holding member 1800. In operation, the filter limbs 1604 are biased to expand in an outward radial direction and as such have an inherent tendency to occupy the greatest internal diameter of the filter housing 300 and associated filter holding member 1800. Correspondingly, once the retention member end 1810 of the filter limb 1800 passes through the aperture 1808 of the filter holding member 1800, the biasing tendency of the filter limb will cause the limb to expand and engage the slot 1806 of the filter holding member. In effect, once the filter element attachment limb 1604 is operatively passed through the aperture 1808 and permitted to expand, the filter element 1600 is retained within the filter housing 300. Retention is effectuated because downstream movement of the filter element 1600 in relation to the filter housing 300 will subsequently cause the retention member end 1810 to operatively engage the base 1802 of the filter holding member 1800. Since the retention member end 1810 is larger in dimension (diameter) than the slot 1806, the end is not permitted to pass through the slot and thus the filter holding member retains the filter limb.

Moreover, the filter element 1600 may be subsequently released from the filter housing 300 by compressing the attachment limbs 1604 such that the retention member end 1810 is aligned and permitted to regress back through the aperture 1806 of the filter holding member 1800. The filter element may be compressed and drawn into a catheter using a snaring catheter/snare wire and standard intervascular techniques, as discussed above with reference to FIGS. 11a through 11h.

In FIG. 18b an alternate embodiment of the filer element 1600 and more particularly an alternate retention end member 1810a is illustrated. In this embodiment, the retention end member 1810a is a geometric configuration of the wire used to fabricate the filter limb 1604. The alternate retention end member 1810a comprises at least one hook or curved end that operatively engages the filter holding member 1800. In particular, the alternate retention end member 1810a is sized larger than the slot 1806 and thus is not permitted to pass through the slot when the filter limb is in the deployed state (i.e., expanded). It its contemplated that the retention end member may be configured in any manner or geometric construct. The primary principle is that the retention end member be sized larger than the integral slot 1806 and to prevent any downstream displacement of the filter element while the filter limbs are expanded.

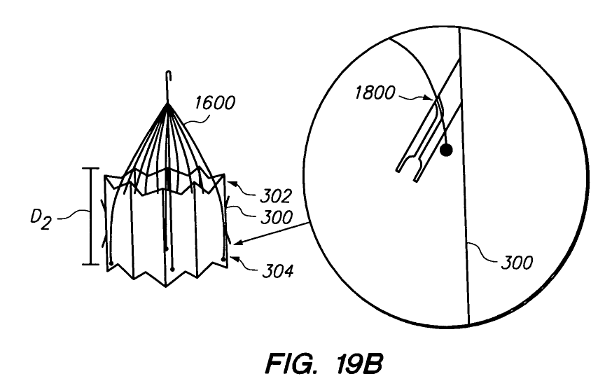

An alternate filter holding member configuration is illustrated in FIGS. 19a and 19b. In these figures, the filter element, filter limb and filter housing are substantially similar to the structure previously disclosed herein. The alternate construction of the filter holding member 1800 in this embodiment is directed towards the open aperture 1900. In contrast to the closed aperture 1808 as previously disclosed with reference to FIGS. 18a and 18b, the open aperture 1900 facilitates the assembly and construction of the IVC vascular filter because the attachment limb and associated retention end member 1810 may be conveniently guided through the open aperture 1900 and into the narrow slot 1806.

As illustrated in a combination of FIGS. 19a and 19b, the insertion depth of the filter element 1600 may be adjusted by the location of the filter holding member 1800 with respect to the filter housing 300. In one embodiment, the filter element may substantially extend beyond a first housing end 302 (FIG. 19a) or conversely the filter element may be substantially located within the filter housing 300 (FIG. 19b). The insertion depth of the filter element 1600 with respect to the filter housing 300 may be adjusted through appropriate placement of the filter holding member 1800. For example, the filter holding member 1800 may be located proximate to the first housing end 302 resulting in the filter element 1600 extending substantially beyond the filter housing 300 and having a minimal insertion depth “D1”. Conversely, the filter holding member 1800 may be located proximate to the second housing end 304 causing the filter element 1600 to be substantially contained within the filter housing 300 having an insertion depth of “D2”. It is further contemplated that any combination or location of the filter holding members may be implemented such that an infinite number of combinations for filter element insertion depths can be established.

It is contemplated that the filter element disclosed herein may be combined with either versions of the filter housing. For example, the bioabsorbable filter housing may be used interchangeably with any filter element, however, the filter element will require the addition of a plurality of filter barbs 1204 (FIG. 12) similar to the securing barbs 400 (FIG. 4) of the filter housing. The primary function of the filter barbs is to retain the filter element in place subsequent to the absorption of the filter housing and filter holding members.

The IVC vascular filter disclosed herein has several advantages over known IVC filters. Firstly, the new vascular filter allows long-term filter removal. In contrast, existing vascular filters are only removable within a predefined time interval that may not be adequate for a specific patient's condition. As a result, if the patient requires vascular filtration for a time period that exceeds the removal time interval of current IVC filters, the filter becomes permanently adhered to the patient's vessel and patent will have the filter for life.

Secondly, the new IVC vascular filter is enabled to transition into a permanent filter by use of the bioabsorbable filter housing. In effect, the new IVC filter with the bioabsorbable housing may be placed for an extended time period (which exceeds current filters) and is completely removable within this time interval. In the event the patient's condition requires a permanent filter, this new IVC filter with bioabsorbable housing, may be left in place and eventually become permanent without any subsequent surgical procedure. Unlike exiting filters, which are only removable for a short duration, the new IVC filter may be removed within a substantially longer time interval. The time interval may be adjusted according to specific bioabsorbable material properties and physical configuration of the filter housing.

Thirdly, the new IVC vascular filter may be configured as a compact filter for use in smaller regions of the circulatory system. Additionally the compact IVC filter may be implemented using either the bioabsorbable or non-absorbable filter housing.

Fourthly, the new IVC vascular filter provides enhanced hemodynamic performance by modifying the orientation and insertion depth of the filter element with respect to the filter housing. As a result the filter will provide increased fluid dynamic performance irrespective of whether the filter element has captured a migrating thrombus.

Finally, another advantage of the new IVC vascular filter is reduced fatigue in the filter element. The filter element used in the present invention is contained in the filter housing in a stress/strain free environment due to the suspended state configuration. The suspended state configuration is obtained by the use of the filter holding members which permit the filter element to float within the filter housing while at the same time being physically constrained within the filter housing. As a result, while the filter housing encapsulates the filter element, the filter housing becomes the stress/strain load path for vascular contractions which in turn removes these forces which would typically be applied to the filter element.

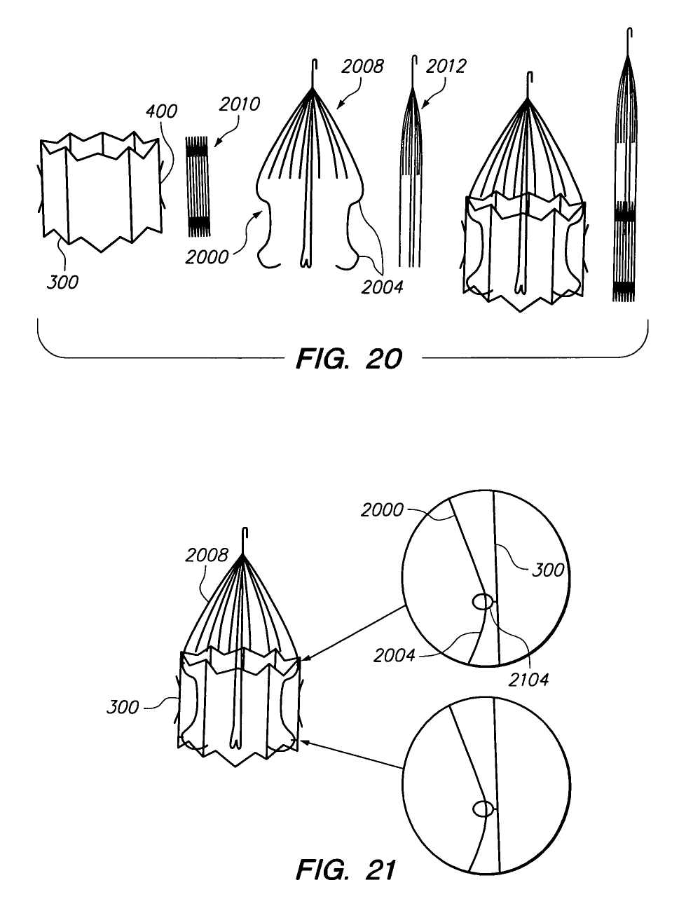

FIG. 20 illustrates an embodiment of the IVC filter 2000 having wherein the limbs 2000 are configured with curves or bends 2004 therein. These attachment limbs for the IVF filter 2008 are different from the prior art in that the limbs 2000 show a complex curve 2004 designed to add stability to the filter when it is within the outer housing unit 300. In this embodiment, as in all embodiments, the housing unit 300 may or may not be configured with barbs 400. View 2010 illustrates the housing in its collapsed state. View 2012 illustrates the filter 2008 it is collapsed state.

The curves 2004 may be configured to provide shallow, neutral resting points so the filter 2008 in the neutral position will be aligned properly with the conical tip centered in the inferior vena cava. Although the curves 2004 are shown in FIG. 20 as located at the end of each lower portion of the limb, it is contemplated that the curves may be located at any area depending on the design of the housing unit 300, filter 2008, and/or location within the body. In addition, various types or shapes of curves 2004 may be utilized to provide structural integrity to the filter 2008 and/or the desired alignment and attachment within the housing unit 300. In one embodiment the shape and location of the curves is selected to securely maintain the filter 2008 within the housing 300 during normal blood flow and when the filter traps a clot or other matter.

FIG. 21 illustrates an alternative embodiment of the filter 2008 and housing 300. This example configuration utilized one or more attachment rings 2104 at either the inferior or the superior end of the housing unit 300, or both. The rings 2104, which may attach to the housing 300, may attach at the curved portion 2004 of the limb 2000. Attachment of the limb 2000 at one or more points along the course of the limb, added stability to the filter. Although the curves 2004 in the limbs 2000 provide stability and aid in the centering the filter 2008, the curves do not interfere with removal of the filter to be easily removed as the limb material is flexible. Although the rings 2104 are shown as fully enclosed rings, in other embodiments the rings may be partially open, or configured as hooks, slots, rails, guides, hook and loop configuration or any other physically configuration capable of achieving the benefits set forth herein. It is contemplated that the engagement between the limb 2000 and the attachment member (modified ring) may be such that the relationship prevents the filter from moving out of the housing, i.e. in the direction of blood flow, and prevents the filter from twisting or spinning radially within the housing 300, but allows filter to be removed from the housing, such as by pulling in the opposite direction of blood flow.

While various embodiments of the invention have been described, it will be apparent to those of ordinary skill in the art that many more embodiments and implementations are possible that are within the scope of this invention. In addition, the various features, elements, and embodiments described herein may be claimed or combined in any configuration or arrangement.

What is claimed is:

1. A vascular filter system comprising:
a resilient filter housing, the filter housing configured with a plurality of filter holding members and a plurality of securing barbs extending outward from the filter housing; and
a resilient filter element, the filter element configured with a plurality of limbs and a retrieval hook, wherein the filter element is sized to fit within the filter housing and the filter element is suspended within the filter housing by each limb engaging at least one filter holding member.

2. The vascular filter system of claim 1, wherein each securing barb is angled outward from the filter housing and toward a first end or a second end of the filter housing.

3. The vascular filter system of claim 1, wherein each filter holding member extends toward a center of the filter housing.

4. The vascular filter system of claim 1, wherein each limb has one or more curves.

5. The vascular filter system of claim 4, wherein each limb extends from a center section of the filter element to a first element end or to a second element end of the filter element, the center section being narrower than the first element end or the second element end.

6. The vascular filter system of claim 4, wherein each limb extends from a second element end to a first element end of the filter element, the second element end being narrower than the first element end.

7. A vascular filter system comprising:
a resilient filter housing, the filter housing comprising:
a plurality of longitudinal support struts connected by transverse angle braces;
at least one filter holding member attached to one or more of the longitudinal support struts or transverse angle braces and extending toward a center of the filter housing; and
at least one securing barb attached to one or more of the longitudinal support struts or transverse angle braces and extending outward from the filter housing; and
a resilient filter element comprising:
a first element end;
a second element end;
a plurality of limbs, each limb having at least one curve; and
a retrieval hook;
wherein the filter element is sized to fit within the filter housing and the filter element is suspended within the filter housing by each limb engaging at least one filter holding member.

8. The vascular filter system of claim 7, wherein the longitudinal support struts and transverse angle braces are arranged to form a cylindrical shape.

9. The vascular filter system of claim 7 further comprising at least one filter barb attached to one or more of the limbs of the filter element and extending outward from the filter element, wherein the filter housing and filter holding members of the filter housing are bioabsorbable.

10. The vascular filter system of claim 7, wherein the filter element is has an hourglass shape with a narrow center section.

11. The vascular filter system of claim 10, wherein the retrieval hook extends from the narrow center section.

12. The vascular filter system of claim 7, wherein the filter element is has ogive shape with a narrow apex distal end.

13. The vascular filter system of claim 12, wherein the retrieval hook extends from the narrow apex distal end.

14. A method for surgically implanting a vascular filter assembly comprising:
accessing a vein and inserting a deployment sheath, wherein the deployment sheath has a vascular filter assembly comprising a filter element and a filter housing disposed within the sheath;
advancing the deployment sheath to a predetermined location;
advancing a deployment member within the deployment sheath until the deployment member contacts the vascular filter assembly;
retracting the deployment sheath while maintaining the position of the deployment member at the predetermined location, wherein retracting the deployment sheath releases the vascular filter assembly allowing the vascular filter assembly to expand within the vein;
removing the deployment sheath and deployment member from the vein.

15. The method of claim 14, wherein the filter assembly further comprises:
a plurality of limbs forming the filter element; and
a plurality of filter holding members attached to the filter housing;
wherein the filter element is suspended within the filter housing by each limb engaging at least one filter holding member.

16. The method of claim 15, wherein the filter housing and at least one filter holding member is bioabsorbable and the filter element further comprises at least one filter barb attached to one or more of the limbs.

17. The method of claim 14 further comprising orienting a filter element having an ogive shape with an apex distal end such that the apex distal end of the filter element is upstream of the limbs when the filter assembly is released from the deployment sheath and into the vein.

18. The method of claim 14, wherein the vascular filter assembly is formed from resilient material.
